(19) United States
(12) Patent Application Publication
(10) Pub. No.: US 2023/0089704 A1
(43) Pub. Date: Mar. 23, 2023

(54) NOVEL COMPOSITION

(71) Applicant: SpecNova LLC, Tysons Corner, VA (US)

(72) Inventor: Sebastian BALCOMBE, Tysons Corner, VA (US)

(73) Assignee: SpecNova LLC, Tysons Corner, VA (US)

(21) Appl. No.: 17/934,408

(22) Filed: Sep. 22, 2022

Related U.S. Application Data
(60) Provisional application No. 63/261,486, filed on Sep. 22, 2021.

(51) Int. Cl. A61K 31/7048; A61K 36/48; A61K 31/58; A61K 31/352; A61K 31/455; A61K 31/706; A23L 33/105; A23L 5/20

(52) U.S. Cl. CPC A61K 31/7048; A61K 36/48; A61K 31/58; A61K 31/352; A61K 31/455; A61K 31/706; A23L 33/105; A23L 5/23; A61K 2236/17; A61K 2236/15; A61K 2236/35; A61K 2236/333; A61K 2236/51; A61K 2236/53; A23V 2002/00

(57) ABSTRACT

The invention provides a fenugreek extract composition containing at least one of diosgenin, gitogenin, sarsasapogenin, dioscin, protodioscin, pseudoprotodioscin, apigenin or a derivative thereof, luteolin or a derivative thereof, nicotinic acid or a derivative thereof, and kaempferol, or a salt or solvate of any one thereof. Also provided are processes of preparing the extract composition, and nutraceutical and pharmaceutical compositions containing the extract composition.

### CROSS REFERENCE TO RELATED APPLICATIONS

This application claims priority to U.S. provisional application No. 63/261,486, filed on Sep. 22, 2021, the entire contents of which are hereby incorporated by reference herein.

### FIELD OF THE INVENTION

The invention relates to a novel fenugreek extract composition and to its various uses as a nutraceutical or pharmaceutical composition. The invention also relates to a novel process for preparing the aforementioned composition.

### BACKGROUND OF THE INVENTION

Fenugreek (Trigonella foenum-graecum) is a clover-like herb native to the Mediterranean region, southern Europe, and western Asia. Fenugreek is used as an ingredient in spice blends and a flavouring agent in foods, beverages, and tobacco. Fenugreek extracts are also used in soaps and cosmetics. In North Africa, Asia, and southern Europe, fenugreek was traditionally used for diabetes and to increase milk supply in women who were breastfeeding. Today, fenugreek is promoted as a dietary supplement for diabetes, menstrual cramps, and other conditions and to stimulate milk production during breastfeeding.

Fenugreek contains many biologically active chemical constituents including steroidal saponins such as the steroidal sapogenin diosgenin.

There is therefore a need to provide alternative fenugreek compositions with improved properties.

### SUMMARY OF THE INVENTION

According to the first aspect of the invention, there is provided a fenugreek extract composition comprising at least one of diosgenin, gitogenin, sarsasapogenin, dioscin, protodioscin, pseudoprotodioscin, apigenin or a derivative thereof, luteolin or a derivative thereof, nicotinic acid or a derivative thereof, and kaempferol, or a salt or solvate of any one thereof.

According to a further aspect of the invention there is provided a nutraceutical composition comprising the extract composition as described herein, and one or more nutraceutically acceptable excipients.

According to a further aspect of the invention there is provided a pharmaceutical composition comprising the extract composition as described herein, and one or more pharmaceutically acceptable excipients.

According to a further aspect of the invention there is provided a process for preparing the composition as described herein, which comprises the steps of:
(a) drying fenugreek seeds;
(b) grinding the material obtained in step (a);
(c) de-fatting the product of step (b) by solvent extraction;
(d) subjecting the product obtained in step (c) to alcohol extraction;
(e) concentrating the product obtained in (d) and dissolving the concentrate in water; and
(f) purifying the product obtained in (e) to yield the claimed composition.

### BRIEF DESCRIPTION OF THE FIGURES

FIG. 1: A representative method of preparation of the claimed fenugreek extract composition.

FIG. 2: Results of a fenugreek supplementation study showing the mean difference in total testosterone from day 0 to day 60 after supplementation of subjects with placebo, 400 mg fenugreek extract and 500 mg fenugreek extract.

FIG. 3: Results of a fenugreek supplementation study showing the mean difference in free testosterone from day 0 to day 60 after supplementation of subjects with placebo, 400 mg fenugreek extract and 500 mg fenugreek extract.

FIG. 4: Results of a fenugreek supplementation study showing the mean difference in estradiol from day 0 to day 30 after supplementation of subjects with placebo or fenugreek extract (400 or 500 mg).

### DETAILED DESCRIPTION OF THE INVENTION

Fenugreek Extract Composition

According to the first aspect of the invention, there is provided a fenugreek extract composition comprising at least one of diosgenin, gitogenin, sarsasapogenin, dioscin, protodioscin, pseudoprotodioscin, apigenin or a derivative thereof, luteolin or a derivative thereof, nicotinic acid or a derivative thereof, and kaempferol, or a salt or solvate of any one thereof.

References herein to “diosgenin” refer to a compound having the following structure:

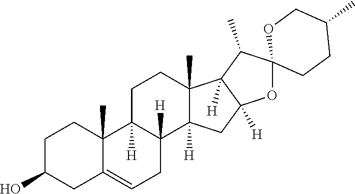

or a salt or solvate thereof.

References herein to “gitogenin” refer to a compound having the following structure:

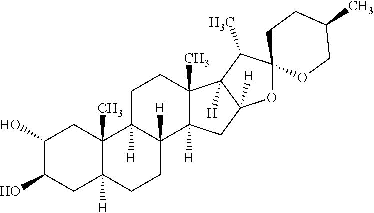

or a salt or solvate thereof.

References herein to “sarsasapogenin” refer to a compound having the following structure:

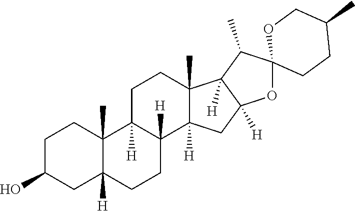

or a salt or solvate thereof.

References herein to “dioscin” refer to a compound having the following structure:

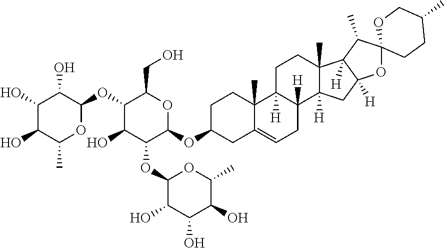

or a salt or solvate thereof.

References herein to “protodioscin” refer to a compound having the following structure:

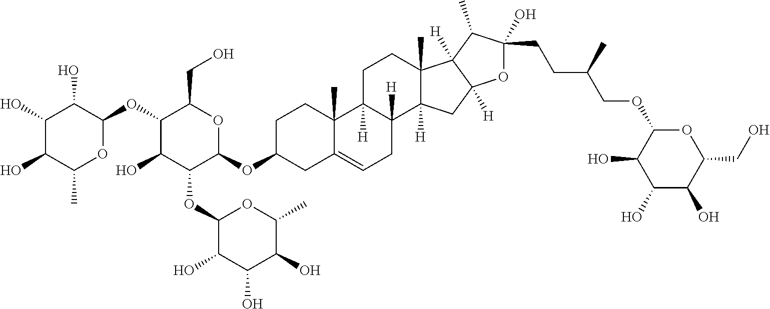

or a salt or solvate thereof.

References herein to “pseudoprotodioscin” refer to a compound having the following structure:

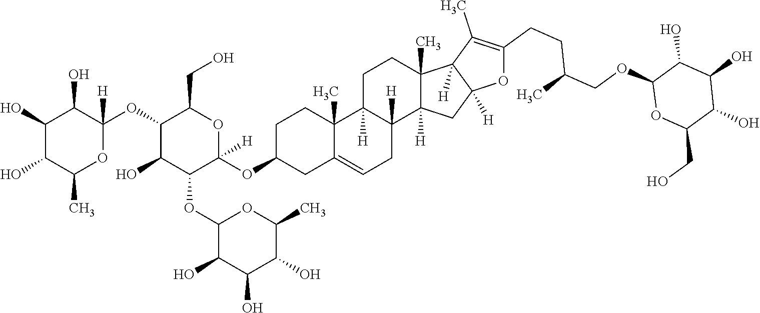

or a salt or solvate thereof.

References herein to “apigenin” refer to a compound having the following structure:

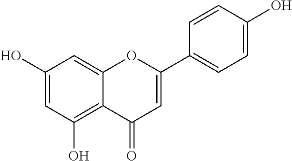 [SMILES: O=c1cc(-c2ccc(O)cc2)oc2cc(O)cc(O)c12]

or a salt or solvate thereof.

References herein to a “derivative of apigenin” refer to any compound which, upon administration to the recipient, is capable of providing (directly or indirectly) apigenin or an active metabolite or residue thereof. In one embodiment, the derivative of apigenin is any one of cosmosiin, apigenin-7-O-(6″-acetyl-beta-D-glucopyranoside), apigenin 7-O-B-D-glucuronide, vitexin, isovitexin, cupressuflavone, and hinokiflavone.

References herein to “cosmosiin” refer to a compound having the following structure:

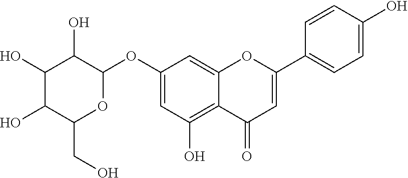 [SMILES: O=c1cc(-c2ccc(O)cc2)oc2cc(OC3OC(CO)C(O)C(O)C3O)cc(O)c12]

or a salt or solvate thereof.

References herein to “apigenin-7-O-(6″-acetyl-beta-D-glucopyranoside)” refer to a compound having the following structure:

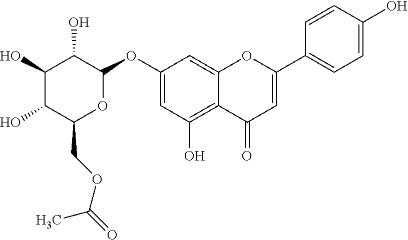

or a salt or solvate thereof.

References herein to “apigenin 7-O-B-D-glucuronide” refer to a compound having the following structure:

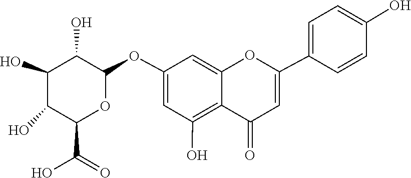

or a salt or solvate thereof.

References herein to “vitexin” refer to a compound having the following structure:

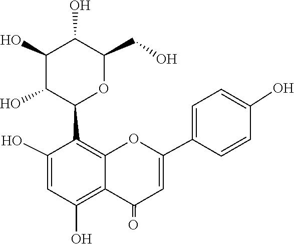

or a salt or solvate thereof.

References herein to “isovitexin” refer to a compound having the following structure:

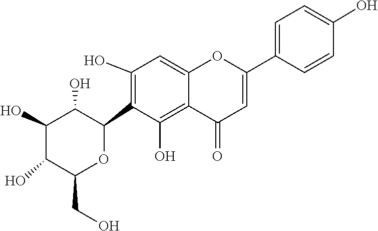 [SMILES: O=c1cc(-c2ccc(O)cc2)oc2cc(O)c([C@H]3O[C@@H](CO)[C@H](O)[C@@H](O)[C@@H]3O)c(O)c12]

or a salt or solvate thereof.

References herein to “cupressuflavone” refer to a compound having the following structure:

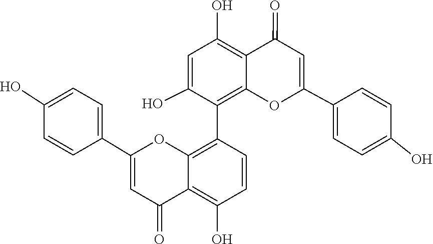 [SMILES: O=c1cc(-c2ccc(O)cc2)oc2c(-c3c(O)cc(O)c4c(=O)cc(-c5ccc(O)cc5)oc34)ccc(O)c12]

or a salt or solvate thereof.

References herein to “hinokiflavone” refer to a compound having the following structure:

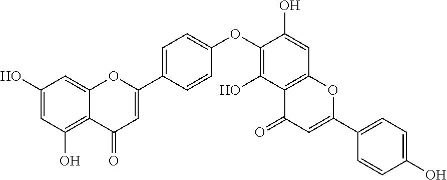 [SMILES: O=c1cc(-c2ccc(Oc3c(O)cc4oc(-c5ccc(O)cc5)cc(=O)c4c3O)cc2)oc2cc(O)cc(O)c12]

or a salt or solvate thereof.

References herein to “luteolin” refer to a compound having the following structure:

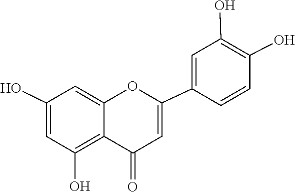 [SMILES: O=c1cc(-c2ccc(O)c(O)c2)oc2cc(O)cc(O)c12]

or a salt or solvate thereof.

References herein to a “derivative of luteolin” refer to any compound which, upon administration to the recipient, is capable of providing (directly or indirectly) luteolin or an active metabolite or residue thereof. In one embodiment, the derivative of luteolin is any one of 3,4,7-trihydroxyflavone, luteolin 5-o-β-d-D-glucopyranoside, neohesperidin and luteolin 7-o-β-d glucopyranose.

References herein to “3,4,7-trihydroxyflavone” refer to a compound having the following structure:

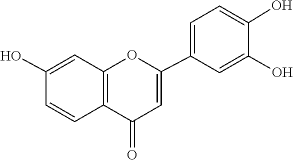 [SMILES: O=c1cc(-c2ccc(O)c(O)c2)oc2cc(O)ccc12]

or a salt or solvate thereof.

References herein to “luteolin 5-o-β-d-D-glucopyranoside” refer to a compound having the following structure:

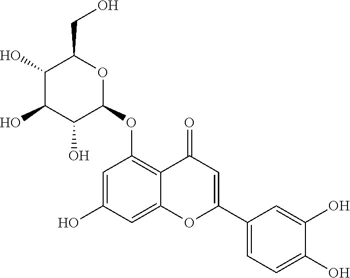 [SMILES: O=c1cc(-c2ccc(O)c(O)c2)oc2cc(O)cc(O[C@@H]3O[C@H](CO)[C@@H](O)[C@H](O)[C@H]3O)c12]

or a salt or solvate thereof.

References herein to “neohesperidin” refer to a compound having the following structure:

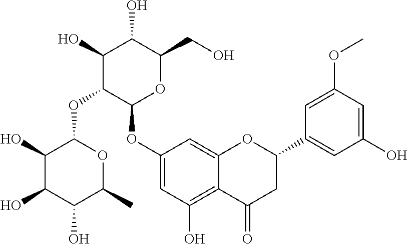 [SMILES: COc1cc(O)cc([C@@H]2CC(=O)c3c(O)cc(O[C@@H]4O[C@H](CO)[C@@H](O)[C@H](O)[C@H]4O[C@@H]4O[C@@H](C)[C@H](O)[C@@H](O)[C@H]4O)cc3O2)c1]

or a salt or solvate thereof.

References herein to “luteolin 7-o-β-d glucopyranose” refer to a compound having the following structure:

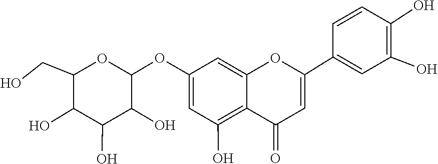 [SMILES: O=c1cc(-c2ccc(O)c(O)c2)oc2cc(OC3OC(CO)C(O)C(O)C3O)cc(O)c12]

or a salt or solvate thereof.

References herein to “nicotinic acid” refer to a compound having the following structure:

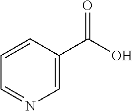

or a salt or solvate thereof.

References herein to a “derivative of nicotinic acid” refer to any compound which, upon administration to the recipient, is capable of providing (directly or indirectly) nicotinic acid or an active metabolite or residue thereof. In one embodiment, the derivative of nicotinic acid is any one of nicotinyl alcohol, inositol nicotinate, and ciclonicate.

References herein to “nicotinyl alcohol” refer to a compound having the following structure:

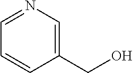

or a salt or solvate thereof.

References herein to “inositol nicotinate” refer to a compound having the following structure:

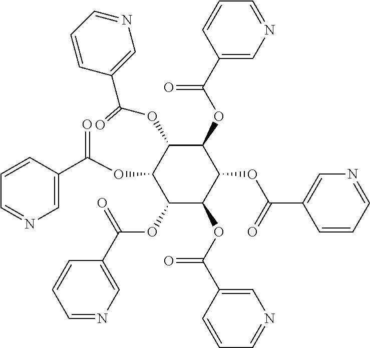 [SMILES: O=C(O[C@H]1[C@H](OC(=O)c2cccnc2)[C@@H](OC(=O)c2cccnc2)[C@H](OC(=O)c2cccnc2)[C@@H](OC(=O)c2cccnc2)[C@H]1OC(=O)c1cccnc1)c1cccnc1]

or a salt or solvate thereof.

References herein to “ciclonicate” refer to a compound having the following structure:

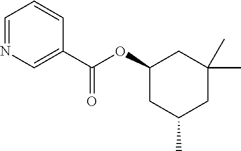 [SMILES: C[C@@H]1C[C@@H](OC(=O)c2cccnc2)CC(C)(C)C1]

or a salt or solvate thereof.

References herein to “kaempferol” refer to a compound having the following structure:

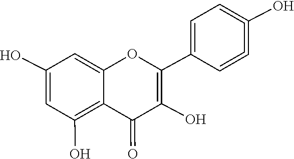

or a salt or solvate thereof.

In one embodiment, the composition comprises at least two of diosgenin, gitogenin, sarsasapogenin, dioscin, protodioscin, pseudoprotodioscin, apigenin or a derivative thereof, luteolin or a derivative thereof, nicotinic acid or a derivative thereof, and kaempferol, or a salt or solvate of any one thereof.

In one embodiment, the composition comprises at least three of diosgenin, gitogenin, sarsasapogenin, dioscin, protodioscin, pseudoprotodioscin, apigenin or a derivative thereof, luteolin or a derivative thereof, nicotinic acid or a derivative thereof, and kaempferol, or a salt or solvate of any one thereof.

In one embodiment, the composition comprises at least four of diosgenin, gitogenin, sarsasapogenin, dioscin, protodioscin, pseudoprotodioscin, apigenin or a derivative thereof, luteolin or a derivative thereof, nicotinic acid or a derivative thereof, and kaempferol, or a salt or solvate of any one thereof.

In one embodiment, the composition comprises at least five of diosgenin, gitogenin, sarsasapogenin, dioscin, protodioscin, pseudoprotodioscin, apigenin or a derivative thereof, luteolin or a derivative thereof, nicotinic acid or a derivative thereof, and kaempferol, or a salt or solvate of any one thereof.

In one embodiment, the composition comprises at least six of diosgenin, gitogenin, sarsasapogenin, dioscin, protodioscin, pseudoprotodioscin, apigenin or a derivative thereof, luteolin or a derivative thereof, nicotinic acid or a derivative thereof, and kaempferol, or a salt or solvate of any one thereof.

In one embodiment, the composition comprises at least seven of diosgenin, gitogenin, sarsasapogenin, dioscin, protodioscin, pseudoprotodioscin, apigenin or a derivative thereof, luteolin or a derivative thereof, nicotinic acid or a derivative thereof, and kaempferol, or a salt or solvate of any one thereof.

In one embodiment, the composition comprises at least eight of diosgenin, gitogenin, sarsasapogenin, dioscin, protodioscin, pseudoprotodioscin, apigenin or a derivative thereof, luteolin or a derivative thereof, nicotinic acid or a derivative thereof, and kaempferol, or a salt or solvate of any one thereof.

In one embodiment, the composition comprises at least nine of diosgenin, gitogenin, sarsasapogenin, dioscin, protodioscin, pseudoprotodioscin, apigenin or a derivative thereof, luteolin or a derivative thereof, nicotinic acid or a derivative thereof, and kaempferol, or a salt or solvate of any one thereof.

In one embodiment, the composition comprises each of diosgenin, gitogenin, sarsasapogenin, dioscin, protodioscin, pseudoprotodioscin, apigenin or a derivative thereof, luteolin or a derivative thereof, nicotinic acid or a derivative thereof, and kaempferol, or a salt or solvate of any one thereof.

In one embodiment, the composition comprises diosgenin, gitogenin, sarsasapogenin, dioscin, protodioscin and pseudoprotodioscin, or a salt or solvate of any one thereof.

In one embodiment, saponins comprise at least 35% (w/w) of said composition.

References to compounds of the invention also include ionic forms, salts, solvates, isomers (including geometric and stereochemical isomers), tautomers, esters, prodrugs, isotopes and protected forms thereof, for example, as discussed below; preferably, the salts or tautomers or isomers or solvates thereof; and more preferably, the salts or tautomers or solvates thereof, even more preferably the salts or tautomers or solvates thereof. Hereinafter, compounds and their ionic forms, salts, solvates, isomers (including geometric and stereochemical isomers), tautomers, esters, prodrugs, isotopes and protected forms thereof as defined in any aspect of the invention (except intermediate compounds in chemical processes) are referred to as “compounds of the invention”.

Salts

Certain compounds of the invention can exist in the form of salts, for example acid addition salts or, in certain cases salts of organic and inorganic bases such as carboxylate, sulfonate and phosphate salts. All such salts are within the scope of this invention, and references to compounds of the invention include the salt forms of the compounds.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, non-aqueous media such as ether, ethyl acetate, ethanol, isopropanol, or acetonitrile are used.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, methanesulfonic (mesylate), ethanesulfonic, naphthalenesulfonic, valeric, acetic, propanoic, butanoic, malonic, glucuronic and lactobionic acids. One particular salt is the hydrochloride salt.

The compounds of the invention may exist as mono- or di-salts depending upon the pKₐ of the acid from which the salt is formed.

It will be appreciated that for use in medicine the salts of the compounds of the invention should be pharmaceutically acceptable. Suitable pharmaceutically acceptable salts will be apparent to those skilled in the art. Pharmaceutically acceptable salts include those described by Berge, Bighley and Monkhouse, J. Pharm. Sci. 1977, 66, pp. 1-19. Such pharmaceutically acceptable salts include acid addition salts formed with inorganic acids e.g. hydrochloric, hydrobromic, sulfuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates may be used, for example in the isolation of compounds of the invention and are included within the scope of this invention. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

Certain of the compounds of the invention may form acid addition salts with one or more equivalents of the acid. The present invention includes within its scope all possible stoichiometric and non-stoichiometric forms.

Solvates

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Pharmaceutically acceptable solvates of the compounds of the invention are within the scope of the invention. In one embodiment, the pharmaceutically acceptable solvates of the compounds of the invention include the hydrate thereof.

Prodrugs

It will be appreciated by those skilled in the art that certain protected derivatives of compounds of the invention, which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. All such prodrugs of compounds of the invention are included within the scope of the invention.

Examples of pro-drug functionality suitable for the compounds of the present invention are described in Drugs of Today, 19, 9, 1983, 499-538 and in Topics in Chemistry, Chapter 31, pp. 306-316 and in “Design of Prodrugs” by H. Bundgaard, Elsevier, 1985, Chapter 1. It will further be appreciated by those skilled in the art, that certain moieties, known to those skilled in the art as “pro-moieties”, for example as described by H. Bundgaard in “Design of Prodrugs” may be placed on appropriate functionalities when such functionalities are present within compounds of the invention.

Certain specific examples of pro-drugs include sulphonated, glucuronidated, methylated, esterificated, acetylated, glutathionated and glycine conjugated derivatives of the compounds of the invention.

Also included within the scope of the compounds and various salts of the invention are polymorphs thereof.

Enantiomers

Where chiral centres are present in compounds of the invention, the present invention includes within its scope all possible enantiomers and diastereoisomers, including mixtures thereof. The different isomeric forms may be separated or resolved one from the other by conventional methods, or any given isomer may be obtained by conventional synthetic methods or by stereospecific or asymmetric syntheses. The invention also extends to any tautomeric forms or mixtures thereof.

Isotopes

The subject invention also includes all pharmaceutically acceptable isotopically-labelled compounds which are identical to those recited in the compounds of the invention but for the fact that one or more atoms are replaced by an atom having an atomic mass or mass number different from the atomic mass or mass number most commonly found in nature.

Examples of isotopes suitable for inclusion in the compounds of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁶N, oxygen, such as ¹⁵O, ¹⁷O and ¹⁸O, phosphorus, such as ³²P, and sulfur, such as ³⁶S.

Certain isotopically-labelled compounds of the invention, for example, those incorporating a radioactive isotope, are useful in drug and/or substrate tissue distribution studies. The compounds of the invention can also have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase) etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹O, ¹⁸F, ¹⁵O and ¹³N, can be useful in Positron Emission Topography (PET) studies for examining target occupancy.

Isotopically-labelled compounds of the invention can generally be prepared by conventional techniques known to those skilled in the art using appropriate isotopically-labelled reagents in place of the non-labelled reagent previously employed.

Purity

Since the compounds of the invention are each preferably provided in substantially pure form, for example at least 60% pure, more suitably at least 75% pure and preferably at least 85%, especially at least 98% pure (% are given on a weight for weight basis). Impure preparations of the compounds may be used for preparing the more pure forms used in the composition.

Nutraceutical Compositions

References herein to a nutraceutical refer to a food, food product, food additive or dietary supplement that provides health and/or medical benefits, such as preventing, treating and enhancing mammalian (e.g. human) conditions. References herein to food extend equally to a drink or beverage comprising said nutraceutical.

According to a further aspect of the invention there is provided a nutraceutical composition comprising the extract composition as described herein, and one or more nutraceutically acceptable excipients.

In one embodiment, the nutraceutical composition additionally comprises one or more additional active ingredients.

In one embodiment, the nutraceutical composition is a tablet or capsule.

In one embodiment, the nutraceutical composition is a food or beverage selected from: water, milk, coffee, tea, juice, protein shake, energy drink, yoghurt and cereal or chocolate bar.

In one embodiment, the nutraceutical composition is for use as a food, food product, food additive or dietary supplement.

The nutraceutically acceptable excipient(s) can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions. Examples of excipients for various types of nutraceutical compositions are set out in more detail below.

The term “nutraceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Nutraceutical compositions containing compounds of the invention can be formulated in accordance with known techniques, see for example, Remington's Pharmaceutical Sciences, Mack Publishing Company, Easton, Pa., USA.

The nutraceutical compositions can be administered to the subject in need thereof in any suitable and convenient form. Suitably, said administration will be orally or topically.

Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as sunflower oil, safflower oil, corn oil or olive oil), and injectable organic esters such as ethyl oleate. Proper fluidity can be maintained, for example, by the use of thickening or coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol, sorbic acid, and the like. It may also be desirable to include agents to adjust tonicity such as sugars, sodium chloride, and the like.

Nutraceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

Thus, tablet compositions can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

Tablets may be designed to release the active compound either upon contact with stomach fluids (immediate release tablets) or to release in a controlled manner (controlled release tablets) over a prolonged period of time or with a specific region of the GI tract.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g; tablets, capsules etc.) can be coated or un-coated. Coatings may act either as a protective film (e.g. a polymer, wax or varnish) or as a mechanism for controlling drug release or for aesthetic or identification purposes. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum, duodenum, jejenum or colon.

Instead of, or in addition to, a coating, the active compound can be presented in a solid matrix comprising a release controlling agent, for example a release delaying agent which may be adapted to release the compound in a controlled manner in the gastrointestinal tract. Alternatively the drug can be presented in a polymer coating e.g. a polymethacrylate polymer coating, which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. In another alternative, the coating can be designed to disintegrate under microbial action in the gut. As a further alternative, the active compound can be formulated in a delivery system that provides osmotic control of the release of the compound. Osmotic release and other delayed release or sustained release formulations (for example formulations based on ion exchange resins) may be prepared in accordance with methods well known to those skilled in the art.

The compounds of the invention may be formulated with a carrier and administered in the form of nanoparticles, the increased surface area of the nanoparticles assisting their absorption. In addition, nanoparticles offer the possibility of direct penetration into the cell. Nanoparticle drug delivery systems are described in “Nanoparticle Technology for Drug Delivery”, edited by Ram B Gupta and Uday B. Kompella, Informa Healthcare, ISBN 9781574448573, published 13 Mar. 2006. Nanoparticles for drug delivery are also described in J. Control. Release, 2003, 91 (1-2), 167-172, and in Sinha et al., Mol. Cancer Ther. August 1, (2006) 5, 1909.

The nutraceutical compositions typically comprise from approximately 1% (w/w) to approximately 95% (w/w) active ingredient and from 99% (w/w) to 5% (w/w) of a nutraceutically acceptable excipient or combination of excipients. Particularly, the compositions comprise from approximately 20% (w/w) to approximately 90% (w/w) active ingredient and from 80% (w/w) to 10% of a nutraceutically acceptable excipient or combination of excipients. The nutraceutical compositions comprise from approximately 1% to approximately 95%, particularly from approximately 20% to approximately 90%, active ingredient.

The nutraceutically acceptable excipient(s) can be selected according to the desired physical form of the formulation and can, for example, be selected from diluents (e.g solid diluents such as fillers or bulking agents; and liquid diluents such as solvents and co-solvents), disintegrants, buffering agents, lubricants, flow aids, release controlling (e.g. release retarding or delaying polymers or waxes) agents, binders, granulating agents, pigments, plasticizers, antioxidants, preservatives, flavouring agents, taste masking agents, tonicity adjusting agents and coating agents.

The skilled person will have the expertise to select the appropriate amounts of ingredients for use in the formulations. For example tablets and capsules typically contain 0-20% disintegrants, 0-5% lubricants, 0-5% flow aids and/or 0-99% (w/w) fillers/or bulking agents (depending on drug dose). They may also contain 0-10% (w/w) polymer binders, 0-5% (w/w) antioxidants, 0-5% (w/w) pigments. Slow release tablets would in addition contain 0-99% (w/w) release-controlling (e.g. delaying) polymers (depending on dose). The film coats of the tablet or capsule typically contain 0-10% (w/w) polymers, 0-3% (w/w) pigments, and/or 0-2% (w/w) plasticizers.

Nutraceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into a polymer or waxy matrix that allow the active ingredients to diffuse or be released in measured amounts.

The compounds of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

This invention also provides solid dosage forms comprising the solid solution described above. Solid dosage forms include tablets, capsules, chewable tablets and dispersible or effervescent tablets. Known excipients can be blended with the solid solution to provide the desired dosage form. For example, a capsule can contain the solid solution blended with (a) a disintegrant and a lubricant, or (b) a disintegrant, a lubricant and a surfactant. In addition a capsule can contain a bulking agent, such as lactose or microcrystalline cellulose. A tablet can contain the solid solution blended with at least one disintegrant, a lubricant, a surfactant, a bulking agent and a glidant. A chewable tablet can contain the solid solution blended with a bulking agent, a lubricant, and if desired an additional sweetening agent (such as an artificial sweetener), and suitable flavours. Solid solutions may also be formed by spraying solutions of drug and a suitable polymer onto the surface of inert carriers such as sugar beads (‘non-pareils’). These beads can subsequently be filled into capsules or compressed into tablets.

Compositions for topical use and nasal delivery include ointments, creams, sprays, patches, gels, liquid drops and inserts (for example intraocular inserts). Such compositions can be formulated in accordance with known methods.

The compounds of the invention will generally be presented in unit dosage form and, as such, will typically contain sufficient compound to provide a desired level of activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The active compound will be administered to a patient in need thereof (for example a human or animal patient) in an amount sufficient to achieve the desired effect.

Although it is anticipated that the nutraceutical composition of the invention will be present within a tablet or capsule, it may also be within a food or beverage. Examples of suitable foods or beverages where the nutraceutical compositions may be contained within include: water, milk, coffee, tea, juice, protein shake, energy drink, yoghurt, cereal or chocolate bar, and the like.

Nutraceutical Utility

Data is presented herein which demonstrates that the composition of the present invention reduces estradiol levels in subjects.

It will therefore be appreciated that the nutraceutical composition of the present invention finds utility in any benefit attributed to reducing estradiol levels. Thus, according to a further aspect of the invention there is provided the nutraceutical composition as described herein for use in improving or increasing one or more of the following: sexual desire and well being, sperm production, stamina, strength, workout output, mobility, athletic speed, reaction time, athletic endurance, muscle mass, confidence, protein synthesis, human lactation, potassium levels, motivation and hair growth; or reduction of one or more of the following: fatigue, cholesterol levels, triglyceride levels, high blood sugar levels, calcification processes in the renal tissue, thyroid hormone levels, growth of cancer cells and menstrual cramps.

Pharmaceutical Compositions

According to a further aspect of the invention there is provided a pharmaceutical composition comprising the extract composition as described herein, and one or more pharmaceutically acceptable excipients.

In one embodiment, the pharmaceutical composition additionally comprises one or more additional active ingredients.

The pharmaceutically acceptable excipient(s) can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions. Examples of excipients for various types of pharmaceutical compositions are set out in more detail below.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Pharmaceutical compositions containing compounds of the invention can be formulated in accordance with known techniques, see for example, Remington's Pharmaceutical Sciences, Mack Publishing Company, Easton, Pa., USA.

The pharmaceutical compositions can be in any form suitable for oral, parenteral, topical, intranasal, intrabronchial, sublingual, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump or syringe driver.

Pharmaceutical formulations adapted for parenteral administration include aqueous and non-aqueous sterile injection solutions which may contain anti-oxidants, buffers, bacteriostats, co-solvents, surface active agents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

The formulations may be presented in unit-dose or multi-dose containers, for example sealed ampoules, vials and prefilled syringes, and may be stored in a freeze-dried (lyophilised) condition requiring only the addition of the sterile liquid carrier, for example water for injections, immediately prior to use. In one embodiment, the formulation is provided as an active pharmaceutical ingredient in a bottle for subsequent reconstitution using an appropriate diluent.

The pharmaceutical formulation can be prepared by lyophilising a compound of the invention. Lyophilisation refers to the procedure of freeze-drying a composition. Freeze-drying and lyophilisation are therefore used herein as synonyms.

Extemporaneous injection solutions and suspensions may be prepared from sterile powders, granules and tablets.

Pharmaceutical compositions of the present invention for parenteral injection can also comprise pharmaceutically acceptable sterile aqueous or non-aqueous solutions, dispersions, suspensions or emulsions as well as sterile powders for reconstitution into sterile injectable solutions or dispersions just prior to use.

Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as sunflower oil, safflower oil, corn oil or olive oil), and injectable organic esters such as ethyl oleate. Proper fluidity can be maintained, for example, by the use of thickening or coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol, sorbic acid, and the like. It may also be desirable to include agents to adjust tonicity such as sugars, sodium chloride, and the like. Prolonged absorption of the injectable pharmaceutical form may be brought about by the inclusion of agents which delay absorption such as aluminum monostearate and gelatin.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

Thus, tablet compositions can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

Tablets may be designed to release the drug either upon contact with stomach fluids (immediate release tablets) or to release in a controlled manner (controlled release tablets) over a prolonged period of time or with a specific region of the GI tract.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g; tablets, capsules etc.) can be coated or un-coated. Coatings may act either as a protective film (e.g. a polymer, wax or varnish) or as a mechanism for controlling drug release or for aesthetic or identification purposes. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum, duodenum, jejenum or colon.

Instead of, or in addition to, a coating, the drug can be presented in a solid matrix comprising a release controlling agent, for example a release delaying agent which may be adapted to release the compound in a controlled manner in the gastrointestinal tract. Alternatively the drug can be presented in a polymer coating e.g. a polymethacrylate polymer coating, which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. In another alternative, the coating can be designed to disintegrate under microbial action in the gut. As a further alternative, the active compound can be formulated in a delivery system that provides osmotic control of the release of the compound. Osmotic release and other delayed release or sustained release formulations (for example formulations based on ion exchange resins) may be prepared in accordance with methods well known to those skilled in the art.

The compounds of the invention may be formulated with a carrier and administered in the form of nanoparticles, the increased surface area of the nanoparticles assisting their absorption. In addition, nanoparticles offer the possibility of direct penetration into the cell. Nanoparticle drug delivery systems are described in “Nanoparticle Technology for Drug Delivery”, edited by Ram B Gupta and Uday B. Kompella, Informa Healthcare, ISBN 9781574448573, published 13 Mar. 2006. Nanoparticles for drug delivery are also described in J. Control. Release, 2003, 91 (1-2), 167-172, and in Sinha et al., Mol. Cancer Ther. August 1, (2006) 5, 1909.

The pharmaceutical compositions typically comprise from approximately 1% (w/w) to approximately 95% (w/w) active ingredient and from 99% (w/w) to 5% (w/w) of a pharmaceutically acceptable excipient or combination of excipients. Particularly, the compositions comprise from approximately 20% (w/w) to approximately 90% (w/w) active ingredient and from 80% (w/w) to 10% of a pharmaceutically acceptable excipient or combination of excipients. The pharmaceutical compositions comprise from approximately 1% to approximately 95%, particularly from approximately 20% to approximately 90%, active ingredient. Pharmaceutical compositions according to the invention may be, for example, in unit dose form, such as in the form of ampoules, vials, suppositories, pre-filled syringes, dragées, tablets or capsules.

The pharmaceutically acceptable excipient(s) can be selected according to the desired physical form of the formulation and can, for example, be selected from diluents (e.g solid diluents such as fillers or bulking agents; and liquid diluents such as solvents and co-solvents), disintegrants, buffering agents, lubricants, flow aids, release controlling (e.g. release retarding or delaying polymers or waxes) agents, binders, granulating agents, pigments, plasticizers, antioxidants, preservatives, flavouring agents, taste masking agents, tonicity adjusting agents and coating agents.

The skilled person will have the expertise to select the appropriate amounts of ingredients for use in the formulations. For example tablets and capsules typically contain 0-20% disintegrants, 0-5% lubricants, 0-5% flow aids and/or 0-99% (w/w) fillers/or bulking agents (depending on drug dose). They may also contain 0-10% (w/w) polymer binders, 0-5% (w/w) antioxidants, 0-5% (w/w) pigments. Slow release tablets would in addition contain 0-99% (w/w) release-controlling (e.g. delaying) polymers (depending on dose). The film coats of the tablet or capsule typically contain 0-10% (w/w) polymers, 0-3% (w/w) pigments, and/or 0-2% (w/w) plasticizers.

Parenteral formulations typically contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into a polymer or waxy matrix that allow the active ingredients to diffuse or be released in measured amounts.

The compounds of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

This invention also provides solid dosage forms comprising the solid solution described above. Solid dosage forms include tablets, capsules, chewable tablets and dispersible or effervescent tablets. Known excipients can be blended with the solid solution to provide the desired dosage form. For example, a capsule can contain the solid solution blended with (a) a disintegrant and a lubricant, or (b) a disintegrant, a lubricant and a surfactant. In addition a capsule can contain a bulking agent, such as lactose or microcrystalline cellulose. A tablet can contain the solid solution blended with at least one disintegrant, a lubricant, a surfactant, a bulking agent and a glidant. A chewable tablet can contain the solid solution blended with a bulking agent, a lubricant, and if desired an additional sweetening agent (such as an artificial sweetener), and suitable flavours. Solid solutions may also be formed by spraying solutions of drug and a suitable polymer onto the surface of inert carriers such as sugar beads (‘non-pareils’). These beads can subsequently be filled into capsules or compressed into tablets.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use and nasal delivery include ointments, creams, sprays, patches, gels, liquid drops and inserts (for example intraocular inserts). Such compositions can be formulated in accordance with known methods.

Examples of formulations for rectal or intra-vaginal administration include pessaries and suppositories which may be, for example, formed from a shaped moldable or waxy material containing the active compound. Solutions of the active compound may also be used for rectal administration.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the invention will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The active compound will be administered to a patient in need thereof (for example a human or animal patient) in an amount sufficient to achieve the desired therapeutic effect.

Therapeutic Utility

According to a further aspect of the invention, there is provided the pharmaceutical composition as defined herein, for use in therapy.

Data is presented herein which demonstrates that the composition of the present invention reduces estradiol levels in subjects. Therefore, according to a further aspect of the invention there is provided the pharmaceutical composition as defined herein, for use in the prophylaxis or treatment of a disease state or condition mediated by estradiol.

Examples of disease states or conditions mediated by estradiol include: infertility, gynecomastia, erectile dysfunction, depression, delayed puberty, fatigue, insomnia, cholestasis, night sweats, and stunted growth.

The compounds of the present invention may be useful for the treatment of the adult population. The compounds of the present invention may be useful for the treatment of the paediatric population.

Process

According to a further aspect of the invention there is provided a process for preparing the composition as described herein, which comprises the steps of:
(a) drying fenugreek seeds;
(b) grinding the material obtained in step (a);
(c) de-fatting the product of step (b) by solvent extraction;
(d) subjecting the product obtained in step (c) to alcohol extraction;
(e) concentrating the product obtained in (d) and dissolving the concentrate in water; and
(f) purifying the product obtained in (e) to yield the claimed composition.

In one embodiment the drying temperature of step (a) is about 50° C.

In one embodiment the solvent used in the solvent extraction of step (c) is a hexane:ethyl acetate solution. In a further embodiment, the ratio of hexane:ethyl acetate is 90:10.

One advantage of performing the solvent extract with hexane:ethyl acetate at a ratio of 90:10 is that it minimizes the molecules such as the strong smelling compounds like sotolon, and the other odor inducing oils that are: Olfactometry diacetyl; 1-Octene-3-one; (Z)-1,5-Octadiene-3-one; 3-isopropyl-2-methoxypyrazine; acetic acid; 3-Isobutyl-2-methoxypyrazine; linalool; butanoic acid; isovaleric acid; caproic acid; eugenol; 3-Amino-4,5-dimethyl-3; 4-dihydro-2(5H)-Furanone; 2,5-dimethylpyrazine; β-pinene; 3-octen-2-one; camphor; terpinen-4-ol; 4-isopropyl-benzaldehyde; neryl acetate and β-caryophyllene.

In one embodiment, the alcohol used in the alcohol extraction of step (d) comprises the use of an ethanol:water mixture. In a further embodiment, the ratio of ethanol:water is 60:40. In another embodiment, the alcohol extraction is performed at a temperature of about 35° C.

In one embodiment, the purification step (f) comprises binding the product obtained in (e) to an adsorption resin. In a further embodiment, the adsorption resin is D101 macroporous adsorption resin. In a further embodiment, the resin bed is washed with 1% ammonia to remove impurities.

In one embodiment, the purification step (f) comprises isolation of the sapogenin/saponin extract. In a further embodiment, the sapogenin/saponin extract is isolated by elution from the resin with ethanol.

In one embodiment, the purification step (f) further comprises isolation of the sugar derivative extract. In a further embodiment, the sugar derivative extract is isolated by elution from the resin with an ethanol:water mixture. In a further embodiment, the ratio of ethanol:water is 60:40.

In one embodiment, the purification step (f) further comprises preparation of a precipitate of the sugar derivative extract. In one embodiment, preparation of a sugar derivative extract precipitate comprises concentrating the sugar derivative extract at a temperature of about 45° C. In a further embodiment, preparation of a sugar derivative extract precipitate comprises precipitating the concentrate with an alcohol. In one embodiment, the alcohol is 95% ethanol.

In one embodiment, the purification step (f) further comprises combining the sapogenin/saponin extract and the sugar derivative extract precipitate to yield the claimed composition.

In one embodiment, the composition is spray dried to obtain an extract powder.

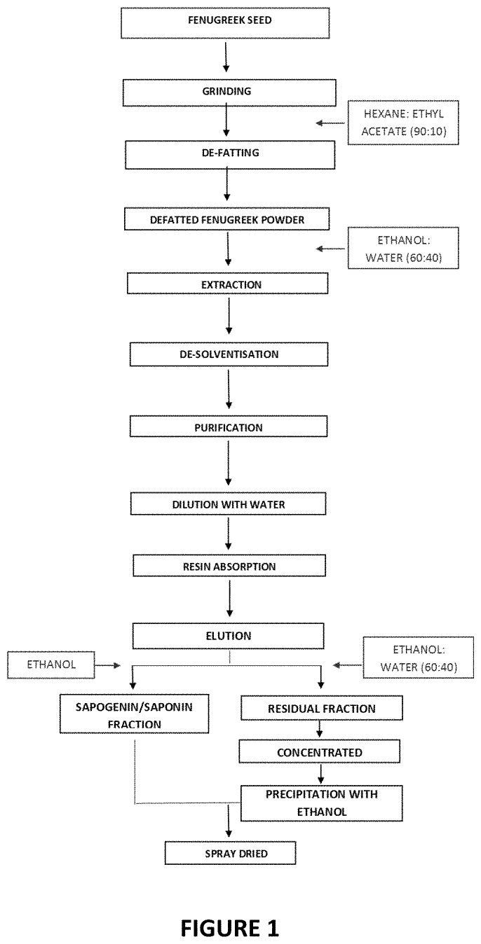

A representative, and non-limiting, method of preparing the fenugreek extract composition of the invention is illustrated by the flowchart in FIG. 1.

The invention will now be described with reference to the following non-limiting examples:

### Example 1: Preparation of Fenugreek Seeds

The fenugreek seeds were washed thoroughly with water to remove dirt and then dried in an air oven at 50° C. for 24 hours. The dried samples were ground into flakes by using a mill at a gap setting of 0.33 mm and its thickness lice around 1.0 mm on average flaking operation. The fenugreek seed flakes were stored in an airtight container, maintaining the moisture content at 10%.

### Example 2: Defatting

100 Kg of fenugreek seed flakes washed with 600 L of hexane:ethyl acetate solution (90:10) by peculation for 8 hours. The washing step was repeated two times. The solvent layer was separated, combined, evaporated and discarded. This solvent extract contained all the fatty acid esters and the volatile fraction of fenugreek. This process yields an odour free fenugreek raw material. After the extraction, the yield was 94-96 Kg.

### Example 3: Extraction

94 Kg of defatted, de-odorized fenugreek powder flake was extracted with 550 L of a solvent mixture of ethanol and water (60:40) for 12 hours at 35° C. by cycling the eluent. The extraction process was repeated three times. The collected extract was then concentrated to a semisolid mass under vacuum at 50° C. The semi-solid mass was then redissolved with deionized water to get a clear solution.

### Example 4: Purification

The clear solution was purified by slowly passing it through a column containing D101 macroporous adsorption resin for 6 hours. The presence of sapogenins and saponins was confirmed by high performance liquid chromatography (HPLC) screening of the eluent. After completion of the column the resin bed was washed with 20 L of 1% ammonia solution to remove the impurities. The resin was desorbed with 8 L of ethanol at a rate of 600 mL per hour. The eluent was collected in separate 2 L volumes and screened for the major saponin molecules using HPLC technique. After the completion of ethanol elution, the resin was desorbed with 25 L of an ethanol:water mixture (60:40). The eluent was collected separately. The ethanol fraction was found to be rich in sapogenins and saponins and the mixed ethanol; water fraction was rich in the sugar derivatives and other high polar bioactive molecules. The ethanol:water fraction containing the sugar derivatives was concentrated at 45° C. to a semisolid mass. This semisolid mass was then precipitated using 95% ethanol. The given precipitate and sapogenin/saponin extract obtained with the ethanol fraction is mixed together and subjected to spray drying process yielding a unique fenugreek extract powder. This extract powder had a total saponin concentration of 35-40% by HPLC and a unique molecular composition of diosgenin, gitogenin, sarsasapogenin, dioscin, protodioscin, pseudoprotodioscin, apigenin and its forms, luteolin and its forms, nicotinic acid and its derivatives, kaempferol identified by liquid chromatography quadrupole time-of-flight mass spectrometry (LC Q-ToF MS).

### Example 5: Efficacy of Fenugreek Supplementation on Healthy Men's Estradiol and Testosterone Levels

Participants (N=57, M age=26.05 years, range=21 to 42 years) were randomized to one of the following three conditions: 400 mg/day fenugreek extract (N=19), 500 mg/day fenugreek extract (N=19), or Placebo control (N=19). Participants completed a self-report, grip strength, and blood assessments at Day 0, Day 30, and Day 60. The supplement was well-tolerated, and no adverse events were reported. Patients in the fenugreek conditions demonstrated increases in total and free testosterone and decreases in estradiol compared to the control group from Day 0 to Day 30 to Day 60; with a dose-response evidenced. Typically, the 500 mg fenugreek condition had larger improvements compared to the 400 mg fenugreek condition.

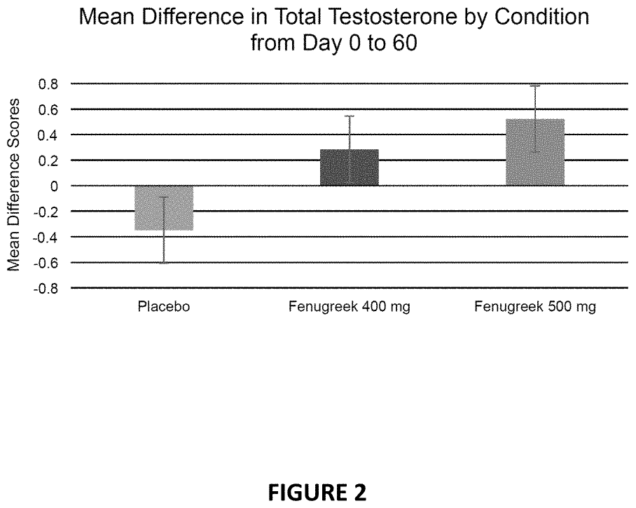

FIG. 2 and Table 1 show the difference in total testosterone (TT) by condition from day 0 to 60. The placebo group had a decrease (M=−0.34) in TT levels compared to an increase in TT for the fenugreek conditions (M=0.40). A dose-response was evidenced in which the 500 mg condition (M=0.52) had a larger increase in TT compared to the 400 mg condition (M=0.28). Therefore, there were significant group differences from Day 0 to Day 60, F(2,48)=3.71, p=0.05. compared to placebo.

TABLE 1
Percentage Change in Total
Testosterone by Condition from Day 0 to 60

| Condition | Day 0 to Day 60 (%) Change |
|---|---|
| Placebo | 0 |
| 400 mg/day fenugreek extract | +3.1 |
| 500 mg/day fenugreek extract | +7.9 |
| Combined fenugreek | +5 |

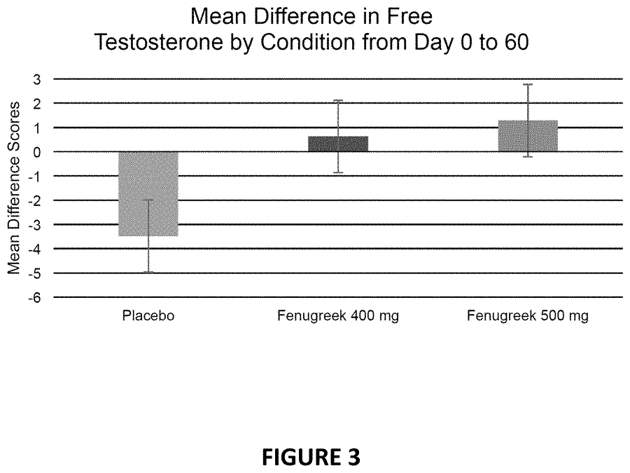

FIG. 3 and Table 2 show the difference in free testosterone (FT) by condition from day 0 to 60. The placebo group had a nonsignificant decrease (M=−3.48) in FT levels compared to an increase in FT for the fenugreek conditions (M=0.93), F(2,48)=2.23, p=0.10. A dose-response was evidenced in which the 500 mg condition (M=1.28) had a larger increase in FT compared to the 400 mg condition (M=0.62).

TABLE 2
Percentage Change in Free
Testosterone by Condition from Day 0 to 60

| Condition | Day 0 to Day 60 (%) Change |
|---|---|
| Placebo | −11.3 |
| 400 mg/day fenugreek extract | +1.6 |
| 500 mg/day fenugreek extract | +9.8 |
| Combined fenugreek | +5.4 |

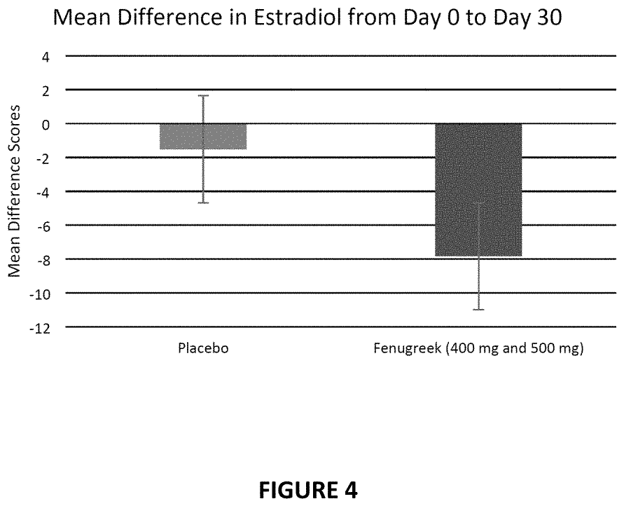

FIG. 4 and Table 3 show the difference in estradiol from day 0 to day 30 and day 60. Combined fenugreek conditions had a significant improvement in estradiol levels (i.e., 15.7% decrease) compared to the placebo from Day 0 to Day 30, F(1,48)=5.15, p=0.03 and was approaching significance, F(2,47)=2.76, p=0.06. at day 0 to 60.

TABLE 3
Percentage Change in Estradiol from Day 0 to Day 30 and Day 60

| | Day 0 to Day 30 (%) |
|---|---|
| Condition | Change |
| Placebo | +8.7 |
| 400 mg/day fenugreek extract | −12 |
| 500 mg/day fenugreek extract | −19.6 |
| Combined fenugreek | −15.7 |

1. A fenugreek extract composition comprising at least one of diosgenin, gitogenin, sarsasapogenin, dioscin, protodioscin, pseudoprotodioscin, apigenin or a derivative thereof, luteolin or a derivative thereof, nicotinic acid or a derivative thereof, and kaempferol, or a salt or solvate of any one thereof.

2. The composition according to claim 1 comprising each of diosgenin, gitogenin, sarsasapogenin, dioscin, protodioscin, pseudoprotodioscin, apigenin or a derivative thereof, luteolin or a derivative thereof, nicotinic acid or a derivative thereof, and kaempferol, or a salt or solvate of any one thereof.

3. The composition according to claim 1, wherein the derivative of apigenin is any one of cosmosiin, apigenin-7-O-(6″-acetyl-beta-D-glucopyranoside), apigenin 7-O-B-D-glucuronide, vitexin, isovitexin, cupressuflavone, and hinokiflavone.

4. The composition according to claim 1, wherein the derivative of luteolin is any one of 3,4,7-trihydroxyflavone, luteolin 5-o-β-d-D-glucopyranoside, neohesperidin and luteolin 7-o-β-d glucopyranose.

5. The composition according to claim 1, wherein the derivative of nicotinic acid is any one of nicotinyl alcohol, inositol nicotinate, and ciclonicate.

6. The composition according to claim 1 comprising diosgenin, gitogenin, sarsasapogenin, dioscin, protodioscin and pseudoprotodioscin, or a salt or solvate of any one thereof.

7. The composition according to claim 1 wherein said saponins comprise at least 35% (w/w) of said composition.

8. A nutraceutical composition comprising the extract composition according to claim 1 and one or more nutraceutically acceptable excipients.

9. The nutraceutical composition according to claim 8, which additionally comprises one or more additional active ingredients.

10. The nutraceutical composition according to claim 8, which is a tablet or capsule.

11. The nutraceutical composition according to claim 9, which is a food or beverage selected from: water, milk, coffee, tea, juice, protein shake, energy drink, yoghurt and cereal or chocolate bar.

12. A food, food product, food additive or dietary supplement comprising the nutraceutical composition according to claim 8.

13. A method of improving or increasing one or more of the following: sexual desire and wellbeing, sperm production, stamina, strength, workout output, mobility, athletic speed, reaction time, athletic endurance, muscle mass, confidence, protein synthesis, human lactation, potassium levels, motivation and hair growth; or reduction of one or more of the following: fatigue, cholesterol levels, triglyceride levels, high blood sugar levels, calcification processes in the renal tissue, thyroid hormone levels, growth of cancer cells and menstrual cramps, said method comprising administering to a subject the nutraceutical composition according to claim 8.

14. A pharmaceutical composition comprising the extract composition according to claim 1 and one or more pharmaceutically acceptable excipients.

15. The pharmaceutical composition according to claim 14, in combination with one or more therapeutic agents.

16. A method for the treatment of a disease state or condition mediated by estradiol, said method comprising administering to a subject the pharmaceutical composition according to claim 14.

17. The method according to claim 16, wherein the disease state or condition mediated by estradiol is selected from: infertility, gynecomastia, erectile dysfunction, depression, delayed puberty, fatigue, insomnia, cholestasis, night sweats, and stunted growth.

18. A process for preparing the composition of claim 1, which comprises the steps of:
(a) drying fenugreek seeds;
(b) grinding the material obtained in step (a);
(c) de-fatting the product of step (b) by solvent extraction;
(d) subjecting the product obtained in step (c) to alcohol extraction;
(e) concentrating the product obtained in (d) and dissolving the concentrate in water; and
(f) purifying the product obtained in (e) to yield the composition of claim 1.

19. The process of claim 18, wherein the purification step (f) comprises isolation of the saponin extract.

20. The process of claim 19, wherein the purification step (f) further comprises:
isolation of the sugar extract;
preparation of a precipitate of the sugar extract; and
combining the saponin extract and the sugar extract precipitate;
and wherein the composition is spray dried to obtain an extract powder.
